(19) United States
(12) Patent Application Publication
(10) Pub. No.: US 2025/0041413 A1
(43) Pub. Date: Feb. 6, 2025

(54) Multi-Cytokine Surface Loaded Particles and Uses in Stimulating Immune Cells for Therapeutic Applications

(71) Applicant: Emory University, Atlanta, GA (US)

(72) Inventors: Sarwish Rafiq, Dunwoody, GA (US); Erik C. Dreaden, Brookhaven, GA (US); A K M Nawshad Hossain, Atlanta, GA (US); Biaggio Uricoli, Atlanta, GA (US); Heather Lin, Atlanta, GA (US)

(21) Appl. No.: 18/715,402

(22) PCT Filed: Dec. 1, 2022
(86) PCT No.: PCT/US2022/051559

§ 371 Date: May 31, 2024

Related U.S. Application Data
(60) Provisional application No. 63/264,727, filed on Dec. 1, 2021.
(60) Provisional application No. 63/389,402, filed on Jul. 15, 2022.

(51) Int. Cl. A61K 39/00; C07K 14/725; C12N 5/0783

(52) U.S. Cl. CPC A61K 39/4611; A61K 39/4631; C07K 14/7051; C12N 5/0636; C12N 2501/2302; C12N 2501/2315; C12N 2501/51; C12N 2501/515

(57) ABSTRACT

This disclosure relates to libraries of multi-cytokine binding particles or other surfaces and uses in method for screening immune cells for improved immune modulating functionality. In certain embodiments, the libraries are used to identify desirable particles for stimulating immune cells useful for cancer and other immune cell therapies.

### CROSS-REFERENCE TO RELATED APPLICATIONS

This application claims the benefit of U.S. Provisional Application No. 63/264,727 filed Dec. 1, 2021 and U.S. Provisional Application No. 63/389,402 filed Jul. 15, 2022. The entirety of each of these applications is hereby incorporated by reference for all purposes.

### BACKGROUND

Methods of stimulating the expansion of certain subsets of T-cells have the potential to generate a variety of T-cell compositions useful in immunotherapy. The various techniques available for expanding human T-cells have relied primarily on the use of accessory cells and/or exogenous growth factors, such as interleukin-2 (IL-2). A co-stimulatory signal may be delivered to a T-cell population, for example, by exposing the cells to a CD3 ligand and a CD28 ligand attached to a solid phase surface, such as a bead. See June et al. U.S. Pat. No. 5,858,358.

To improve the ability of immune cells to kill cancerous cells, T cells can be isolated from the blood of a patient and transfected with a vector that expresses chimeric antigen receptors (CARs) allowing one to specifically target proteins expressed on the surface. When administered back into the patient, the modified T cells attack the cancerous cells. Several T CAR based therapies are clinically approved for treating certain hematological cancers. However, relapse is not uncommon and CAR T cell therapies are reported to be less effective at treating solid tumors. In addition, low levels of naive T cells in peripheral blood subset sometimes leads to production failures. Harvesting enough T cells is difficult in patients with lymphocytopenia, which commonly develops because of disease progression or previous chemotherapy. Thus, there is a need to identify improvements.

Rafiq et al. report engineering strategies to overcome the current roadblocks in CAR T cell therapy. Nat Rev Clin Oncol. 2020, 17(3):147-167.

Balakrishnan et al. report nanoparticles for enhanced adoptive T cell therapies. Front. Immunol, 2021, 12:600659.

References cited herein are not an admission of prior art.

### SUMMARY

This disclosure relates to libraries of multi-cytokine binding particles or surfaces and uses in methods for screening immune cells for improved immune modulating functionality. In certain embodiments, the libraries are used to identify desirable particles for stimulating immune cells useful for cancer and other immune cell therapies.

In certain embodiments, this disclosure relates to particles, e.g., paramagnetic beads, coated with agents that specifically bind molecular targets. In certain embodiments, this disclosure relates to surfaces with multiple zones coated with agents that specifically bind molecular targets. In certain embodiments, the agents proteins are or are derived from variable light or heavy chain antibody sequences. In certain embodiments, the particles or surfaces are used in methods for identifying optimal cell activation and expansion of immune cells.

In certain embodiments, this disclosure relates to particles or surfaces with zones comprising a substantially homogeneous coating with a specific binding agent that binds CD3, a specific binding agent that binds CD28, and a specific binding agent that binds a first cytokine, wherein the specific binding agent is complexed with the first cytokine. In certain embodiments, the specific binding agent that binds a first cytokine is a specific binding agent that binds IL-2. In certain embodiments, the specific binding agent that binds IL-2 is an anti-IL-2 antibody wherein the anti-IL-2 antibody is binding IL-2 providing an IL-2 antibody binding complex coated on the particle or zone.

In certain embodiments, the coating further comprises a specific binding agent that binds a second cytokine, and the specific binding agent is complexed with the second cytokine, wherein the first cytokine and the second cytokine are not the same molecule, e.g., not the same protein binding sequence. In certain embodiments, the specific binding agent that binds a second cytokine is a specific binding agent that binds IL-15. In certain embodiments, the specific binding agent that binds IL-15 is an extracellular domain of IL-15Ralpha and wherein the extracellular domain of IL-15Ralpha is binding IL-15 providing an IL-15 and IL-15Ralpha binding complex coated on the particle or zone.

In certain embodiments, this disclosure relates to methods of stimulating immune cells comprising contacting particles or zones as disclosed herein with immune cells providing phenotypic change in the immune cells. In certain embodiments, the particles have an average diameter of between about 500 nm to 2000 nm. In certain embodiments, the ratio of cells to particles is about 1:5 or 1:10 or about 1:5 or 1:25. In certain embodiments, the phenotypic change in the immune cell is increased CD62L+ cell expression. In certain embodiments, the phenotypic change in the immune cells is increased clustering. In certain embodiments, the phenotypic change in the immune cells is increased CAR cell expression. In certain embodiments, contacting a particle or zone as reported herein with an immune cell is in in the absence of exogenous IL-2. In certain embodiments, contacting a particle or zone as reported herein with an immune cell is in or in contact with a growth medium.

In certain embodiments, this disclosure relates to methods of treating cancer or other immune cell disease or condition comprising, contacting immune cells with particles or zones as reported herein providing activated immune cells; contacting, transfecting, or inserting into the activated immune cells a recombinant vector expressing a chimeric antigen receptor providing chimeric antigen receptor expressing immune cells; and administering an effective amount of chimeric antigen receptor expressing immune cells to a subject in need thereof.

In certain embodiments, the immune cells are derived from a subject diagnosed with cancer or a cancer free donor. In certain embodiments, the immune cells are obtained from the same subject that is to receive the administration (autologous donor). In certain embodiments, the immune cells are obtained from a subject that is health (allogeneic or syngeneic donor).

In certain embodiments, the cancer is a hematological cancer. In certain embodiments, the cancer is a metastatic cancer. In certain embodiments, the cancer is a tumor. In certain embodiments, administering the activated immune cells are in combination with administering an additional anticancer agent or other active agent.

In certain embodiments, this disclosure relates to libraries of particles or surfaces comprising a plurality of zones, wherein each of the particles or zones are coated with a plurality of specific binding agents that bind CD3, CD28, and a first cytokine, wherein 3, 4, 5, or more of the particles or zones are coated with unique concentrations of specific binding agents on each particle or zone. In certain embodiments, the first cytokine is IL-2, wherein the specific binding agent that binds IL-2 is an anti-IL-2 antibody and wherein the anti-IL-2 antibody is binding IL-2 providing an IL-2 specific binding agent complex coated on the particle or zone. In certain embodiments, the particles or zones further comprise a second cytokine. In certain embodiments, the second cytokine is IL-15, wherein the specific binding agent that binds IL-15 is an extracellular domain of IL-15Ralpha and wherein the extracellular domain of IL-15Ralpha is binding IL-15 providing an IL-15 and IL-15Ralpha binding complex coated on the particles or zones.

In certain embodiments, this disclosure relates to method of screening, identifying or selecting optimal particles or zones for managing cancer or an immune therapy comprising, obtaining a sample comprising immune cells from a subject; contacting the sample with a library of particles or zones as disclosed herein; detecting, measuring, quantifying, or observing a phenotypic change in the immune cells; and selecting particles or zones with an optimal phenotypic change for use in stimulating the immune cells for use in a cancer or immune cell therapy.

In certain embodiments, this disclosure relates to in vitro cell culture compositions comprising particles, zones, and/or cells reported herein.

### BRIEF DESCRIPTION OF THE SEVERAL VIEWS OF THE DRAWINGS

FIG. 1A illustrates the assembly and structure of Multi-Cytokine Backpacks (MCBs). Modular and rapid self-assembly of aCD3×aCD28×aIL-2/IL-2×aIL-15R/IL-15 coated particles occur when attaching human anti-IL-2 IgG and IL-15Rα to protein G-conjugated iron oxide microparticles and subsequent cytokine complexation. T cell interactions provide primary, co-stimulatory, and cytokine signaling for T cell activation and expansion.

FIG. 1B provides data on representative test particle size as measured by dynamic light scattering and transmission electron microscopy. Consistent and reproducible synthesis of MCBs was confirmed between batches by calculating abundance of aCD3 and aIL-2 on each MCB.

FIG. 1C shows data on the concentration of cells yielded by activation with Dynabeads™ (DB, anti-CD3+ and anti-CD28+ coated) and adding exogenous IL-2 (exoIL-2), when compared with MCBs having anti-CD3, anti-CD28, and/or anti-IL-2/IL-2, and/or anti-IL-15/IL-15. Experiments adding exoIL-2, or MCB without exoIL-2 were performed. MCB without exoIL-2 yielded the highest number of cells in culture. Brightfield microscopy reveals increased clustering of MCB-stimulated T cells compared to DB.

FIG. 1D shows data where primary T cells were stimulated with increasing cell-to-bead MCB ratios. The ratio of 1:10 cell:beads was chosen for optimal T cell activation in subsequent experiments. MCBs without exoIL-2 were capable of anti-CD19 CAR transduction comparable to DB and MCB with exo-IL2.

FIG. 1E show data where CAR T cells were manufactured with MCBs (anti-CD3, anti-CD28, anti-IL-2, and anti-IL-15) versus the same protein components (DB+exIL2 and exIL15) in soluble “free protein” added to the cell culture indicating increased CD62L+CAR T cells. Similar experiments indicated the T cells had an increase CD8:CD4 ratio.

FIG. 2A illustrates a library screen of multi-cytokine backpacks to identify top candidates that manufacture less differentiated CAR T cells.

FIG. 2B shows data from multi-dimensional flow cytometry analysis of CARs stimulated with MCBs. The highest ratio, CAR+CD8+CD45RA+CCR7+ include those from wells (#09, #16 #27, #43) Box bars. Dotted line denotes control experiments run using Dynabeads™ (CD3 and CD28 beads) with exogenous IL-2.

FIG. 3A shows data where candidate formulations contained aCD3/aCD28/aIL-2/IL15Ra. MCBs were loaded with the relative protein abundances. MCB formulations were used to manufacture CAR T cells from healthy donor PBMCs. MCB-manufactured CAR T cell demonstrated comparable transduction efficiencies to DB-manufactured CAR T. MCB formulations were capable of increasing CD8:CD4 ratios of CAR T cells compared to DB-manufactured cells.

FIG. 3B shows data where MCBs were used to manufacture CAR T cells from diffuse large B-cell lymphoma (DLBCL) donor patient samples to evaluate phenotypic differences in the setting of disease. Similar to healthy donors, MCBs transduced DLBCL T cells with anti-CD19 CARs increased CD8:CD4 ratios when compared to DB+IL-2.

FIG. 3C shows data indicating MCBs transduced DLBCL CAR T cells provide an increase in CD62L+CAR T cells.

FIG. 4A illustrates experiments performed to determine whether MCB-manufactured CAR T cells have enhanced in vivo anti-tumor efficacy in models. Lymphoma model NSG mice were engrafted with 0.1e6 Raji-GFPffLuc+ cells intravenously (IV). After 4 days, mice were treated with 1e6 anti-CD19 CART cells manufactured with either MCB or DB. Bioluminescence of tumor burden was monitored weekly.

FIG. 4B shows data indicating MCB-manufactured CAR T cell-treated mice enhanced overall survival of mice compared to CAR T cells manufactured with DB.

FIG. 5A illustrates experiments performed to determine whether MCB-manufactured CAR T cells are effective in an ovarian model where SKOV3-Muc16CD+ tumor cells were engrafted in SCID/Beige mice intraperitoneally. Mice were treated with anti-Muc16CD CAR T cells 7 days later via intraperitoneal injection.

FIG. 5B shows data indicating MCB-manufactured CAR T cells enhanced overall survival of mice compared to CAR T cells manufactured with DB. Formulation #27 demonstrated the highest overall survival. These results demonstrate the utility of screening different MCB formulations to manufacture optimal CAR T cells specific for different tumor types.

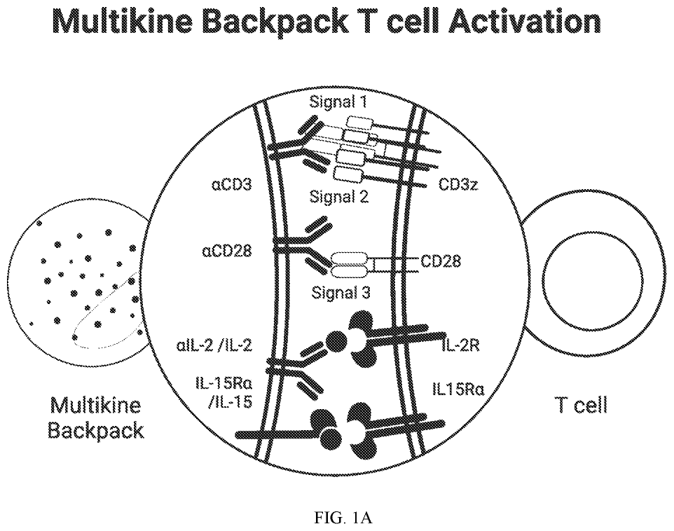

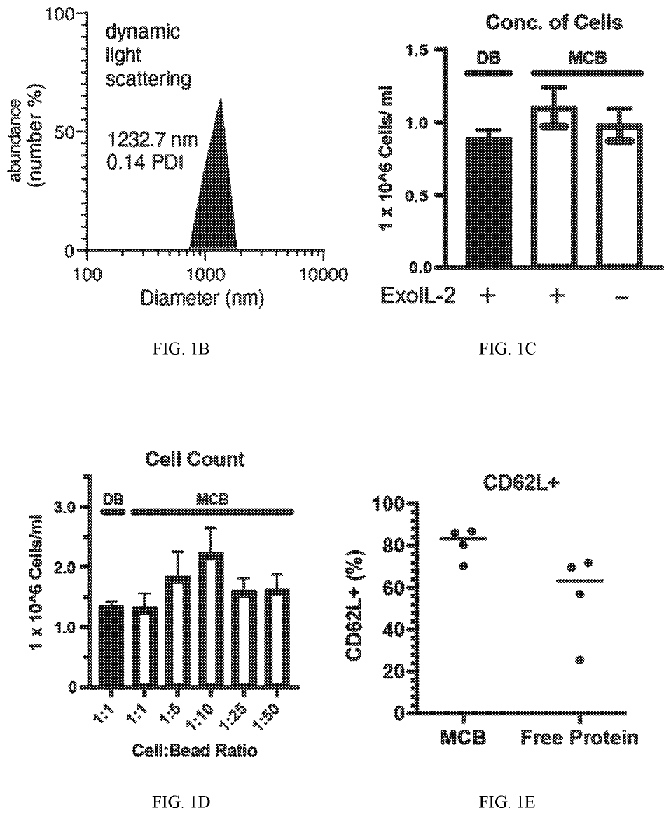

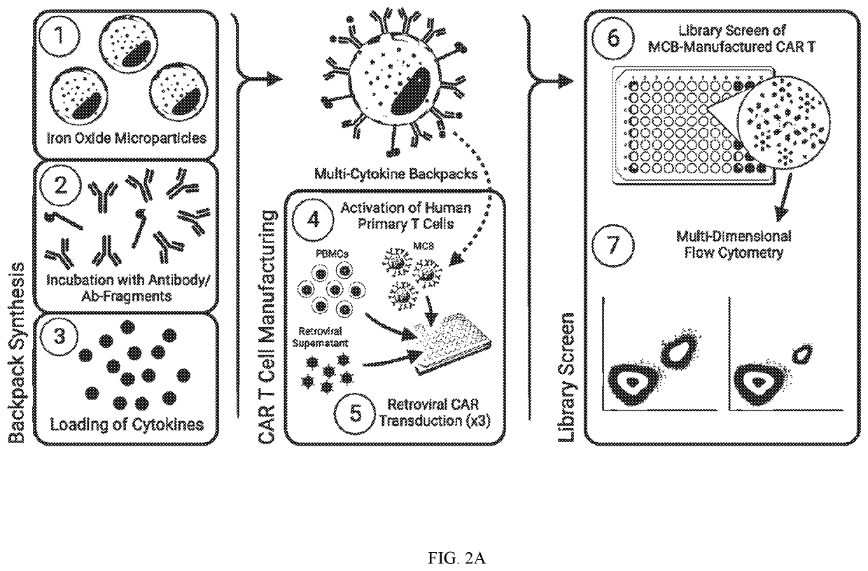

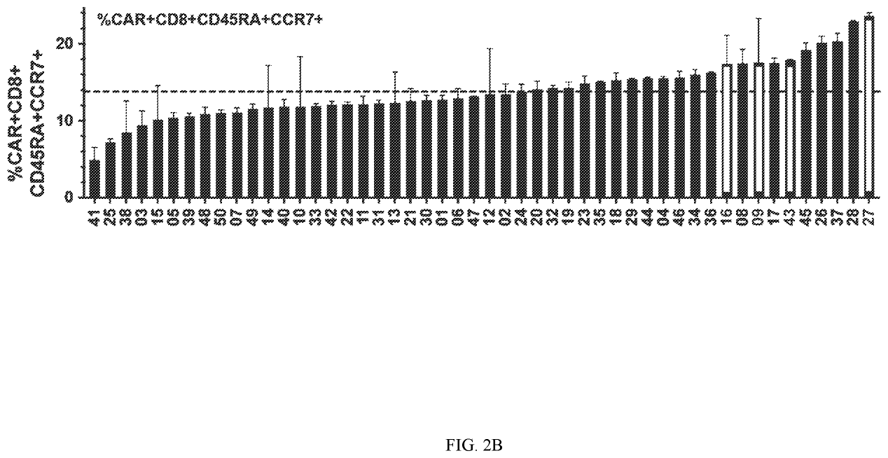

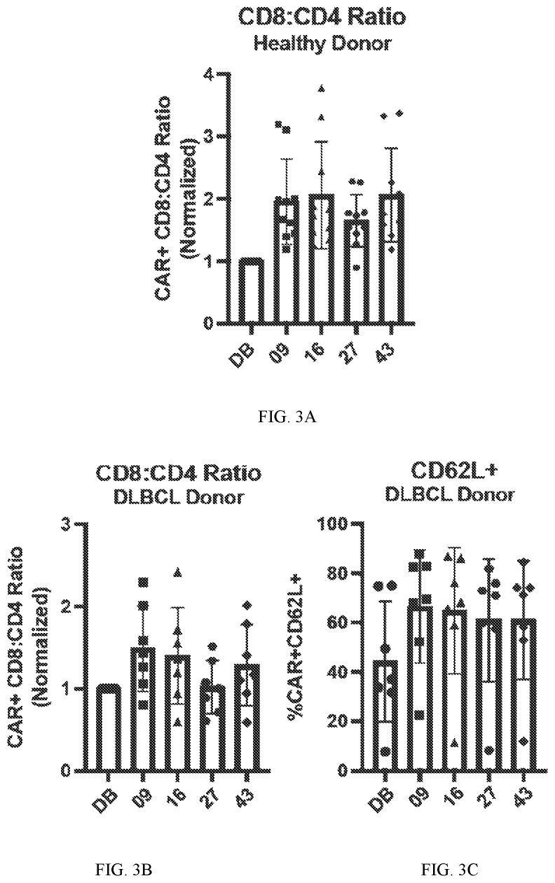

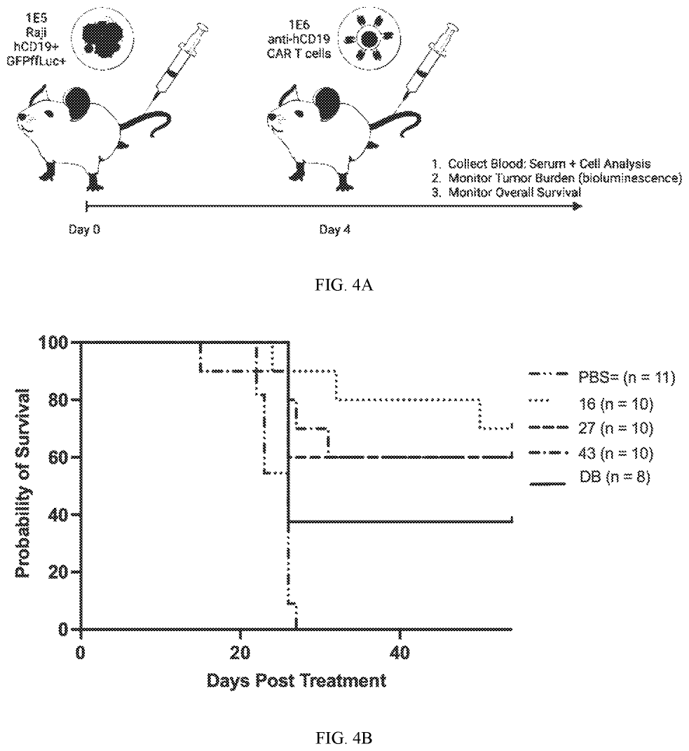

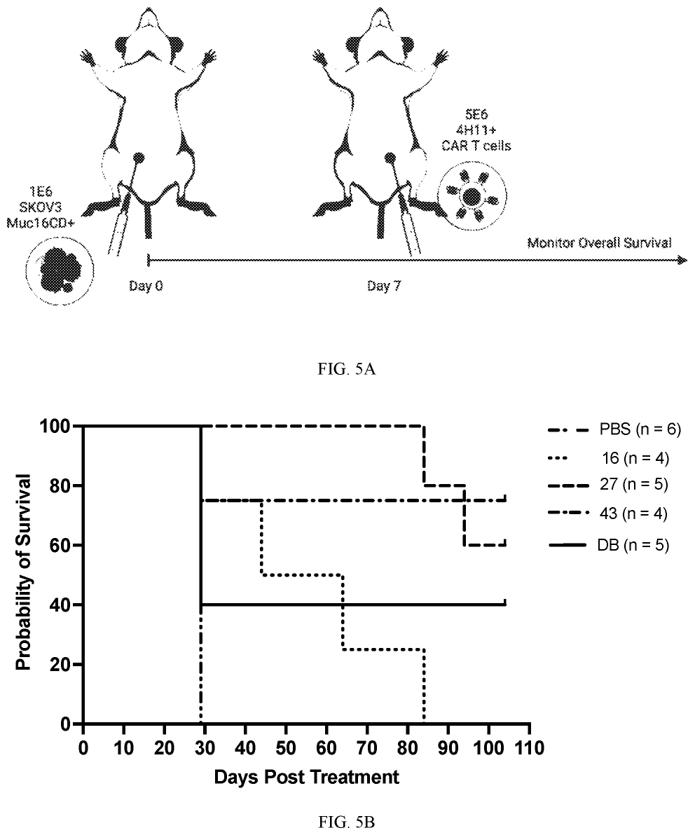

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims. Thus, reference to an “embodiment” refers to an example of the invention and is not necessarily limited by such an example.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of immunology, medicine, organic chemistry, biochemistry, molecular biology, pharmacology, physiology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods.

As used herein, the term “surface” refers to the outside part of an object. The area is typically of greater than about one hundred square nanometers, one square micrometer, or more than one square millimeter. Examples of contemplated surfaces are on a particle/bead, wafer, array, well, microscope slide, transparent or opaque glass, polymer, or metal. A “zone” refers to a segment within a surface that has a unique substantially homogeneous chemical coating. A microtiter plate is a flat plate with multiple “zones” or “wells” which may contain particles or other surfaces for the purpose of creating unique substantially homogeneous chemical coatings.

“Subject” refers any animal, preferably a human patient, livestock, or domestic pet.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term “specific binding agent” refers to a molecule, such as a proteinaceous molecule, that binds a target molecule with a greater affinity than other random molecules or proteins. Examples of specific binding agents include an antibody that bind an epitope of an antigen or a receptor which binds a ligand. In certain embodiments, “Specifically binds” refers to the ability of a specific binding agent (such as an ligand, receptor, enzyme, antibody or binding region/fragment thereof) to recognize and bind a target molecule or polypeptide, such that its affinity (as determined by, e.g., affinity ELISA or other assays) is at least 10 times as great, but optionally 50 times as great, 100, 250 or 500 times as great, or even at least 1000 times as great as the affinity of the same for any other or other random molecule or polypeptide.

As used herein, the term “ligand” refers to an organic molecule, i.e., substantially comprised of carbon, hydrogen, and oxygen, that binds a “receptor.” Receptors are organic molecules typically found on the surface of a cell. Through binding a ligand to a receptor, the cell has a signal of the extra cellular environment which may cause changes inside the cell. As a convention, a ligand is usually used to refer to the smaller of the binding partners from a size standpoint, and a receptor is usually used to refer to a molecule that spatially surrounds the ligand or portion thereof. However as used herein, the terms can be used interchangeably as they generally refer to molecules that are specific binding partners. For example, a glycan may be expressed on a cell surface glycoprotein and a lectin may bind the glycan. As the glycan is typically smaller and surrounded by the lectin during binding, it may be considered a ligand even though it is a receptor of the lectin binding signal on the cell surface. In another example, a double stranded oligonucleotide sequence contains two complimentary nucleic acid sequences. Either of the single stranded sequences may be consider the ligand or receptor of the other. In certain embodiments, a ligand is contemplated to be a compound that has a molecular weight of less than 500 or 1,000. In certain embodiments, a receptor is contemplated to be a compound that has a molecular weight of greater than 2,000 or 5,000.

In certain contexts, an “antibody” refers to a protein-based molecule that is naturally produced by animals in response to the presence of a protein or other molecule or that is not recognized by the animal's immune system to be a “self” molecule, i.e., recognized by the animal to be a foreign molecule, i.e., an antigen to the antibody. The immune system of the animal will create an antibody to specifically bind the antigen, and thereby targeting the antigen for degradation, or any organism attached to the antigen. It is well recognized by skilled artisans that the molecular structure of a natural antibody can be synthesized and altered by laboratory techniques. Recombinant engineering can be used to generate fully synthetic antibodies or fragments thereof providing control over variations of the amino acid sequences of the antibody. Thus, the term “antibody” is intended to include natural antibodies, monoclonal antibodies, or non-naturally produced synthetic antibodies, such as specific binding single chain antibodies, bispecific antibodies, or fragments thereof. These antibodies may have chemical modifications.

From a structural standpoint, a human antibody is a combination of proteins: two heavy chain proteins and two light chain proteins. The heavy chains are longer than the light chains. The two heavy chains typically have the same amino acid sequence. Similarly, the two light chains typically have the same amino acid sequence. Each of the heavy and light chains contain a variable segment that contains amino acid sequences which typically participate in binding to the antigen. The variable segments of the heavy chain do not have the same amino acid sequences as the light chains. The variable segments are often referred to as the antigen binding domains. Recombinantly produced single chains of the variable regions of the heavy chain and/or light chain are often alone sufficient for specific binding to an antigen, although usually to a lesser extent than the complete antibody. The antigen and the variable regions of the antibody may physically interact with each other at specific smaller segments of an antigen often referred to as the “epitope.” Epitopes usually consist of surface groupings of molecules, for example, amino acids or carbohydrates. The terms “variable region,” “antigen binding domain,” and “antigen binding region” refer to that portion of the antibody molecule which contains the amino acid residues that interact with an antigen and confer on the antibody its specificity and affinity for the antigen. Small binding regions within the antigen-binding domain that typically interact with the epitope are also commonly alternatively referred to as the “complementarity-determining regions, or CDRs.”

The term “nucleic acid” refers to a polymer of nucleotides, or a polynucleotide, e.g., RNA, DNA, or a combination thereof. The term is used to designate a single molecule, or a collection of molecules. Nucleic acids may be single stranded or double stranded and may include coding regions and regions of various control elements.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “heterologous” nucleic acid sequence or peptide sequence refers to a nucleic acid sequence or a peptide sequence that does not naturally occur, e.g., because the whole sequence contains a segment from other plants, bacteria, viruses, other organisms, or joinder of two sequences that occur the same organism but are joined together in a manner that does not naturally occur in the same organism or any natural state.

The term “recombinant” when made in reference to a nucleic acid molecule refers to a nucleic acid molecule which is comprised of segments of nucleic acid joined together by means of molecular biological techniques provided that the entire nucleic acid sequence does not occurring in nature, i.e., there is at least one mutation in the overall sequence such that the entire sequence is not naturally occurring even though separately segments may occur in nature. The segments may be joined in an altered arrangement such that the entire nucleic acid sequence from start to finish does not naturally occur. The term “recombinant” when made in reference to a protein or a peptide refers to a protein molecule that is expressed using a recombinant nucleic acid molecule.

The term “vector” refers to a recombinant nucleic acid containing a desired coding sequence and appropriate nucleic acid sequences necessary for the expression of the operably linked coding sequence in a particular host organism or expression system, e.g., cellular or cell-free expression systems. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. In certain embodiments, this disclosure contemplates a vector encoding a peptide disclosed herein in operable combination with a heterologous promoter.

As used herein, a “chimeric antigen receptor” or “CAR” refers to a protein receptor, which introduces an antigen specificity, via an antigen binding domain, onto cells (immune cells) to which it is expressed (for example T cells such as naive T cells, central memory T cells, effector memory T cells or combination thereof) thus combining the antigen binding properties of the antigen binding domain with the T cell activity (e.g. lytic capacity). A CAR typically includes an extracellular antigen-binding domain (ectodomain), a transmembrane domain and an intracellular signaling domain. The intracellular signaling domain generally contains at least one immunoreceptor tyrosine-based activation motif (ITAM) signaling domain, e.g., derived from CD3zeta, and optionally at least one costimulatory signaling domain, e.g., derived from CD28 or 4-1BB.

The terms, “cell culture” or “growth medium” or “media” refers to a composition that contains components that facilitate cell maintenance and growth through protein biosynthesis, such as vitamins, amino acids, inorganic salts, a buffer, and a fuel, e.g., acetate, succinate, a saccharide and/or optionally nucleotides. Typical components in a growth medium include amino acids (histidine, isoleucine, leucine, lysine, methionine, phenylalanine, threonine, tryptophan, valine and others); vitamins such as retinol, carotene, thiamine, riboflavin, niacin, biotin, folate, and ascorbic acid; carbohydrate such as glucose, galactose, fructose, or maltose; inorganic salts such as sodium, calcium, iron, potassium, magnesium, zinc; serum; and buffering agents. Additionally, a growth media may contain phenol red as a pH indication. Components in the growth medium may be derived from blood serum or the growth medium may be serum-free. The growth medium may optionally be supplemented with albumin, lipids, insulin and/or zinc, transferrin or iron, selenium, ascorbic acid, and an antioxidant such as glutathione, 2-mercaptoethanol or 1-thioglycerol. Other contemplated components contemplated in a growth medium include ammonium metavanadate, cupric sulfate, manganous chloride, ethanolamine, and sodium pyruvate. Minimal Essential Medium (MEM) is a term of art referring to a growth medium that contains calcium chloride, potassium chloride, magnesium sulfate, sodium chloride, sodium phosphate and sodium bicarbonate), essential amino acids, and vitamins: thiamine (vitamin B1), riboflavin (vitamin B2), nicotinamide (vitamin B3), pantothenic acid (vitamin B5), pyridoxine (vitamin B6), folic acid (vitamin B9), choline, and myo-inositol (originally known as vitamin B8). Various growth mediums are known in the art. Dulbecco's modified Eagle's medium (DMEM) is a growth medium which contains additional components such as glycine, serine, and ferric nitrate with increased amounts of vitamins and amino acids.

In order to improve the ability of immune cells to kill cancerous cells, T cells can be isolated from the blood of a patient and modified with a recombinant vector to express chimeric antigen receptors (CARs) that specifically target proteins expressed on the surface of cancerous cells and stimulate an immune response. When put back into the patient, the cells attack the cancerous cells. Brentjens et al. report that T cells altered to bind CD19 can induce remissions of cancer in adults with chemotherapy-refractory acute lymphoblastic leukemia. Sci Transl Med, 2013, 5(177):177ra38.

In certain embodiments, the targeting sequence in a chimeric antigen receptor refers to any variety of molecules or polypeptide sequences capable of selectively binding to a targeted associated molecule. The targeting sequences may be derived from variable binding regions of antibodies, single chain antibodies, and antibody mimetics. In certain embodiments, targeting sequence is a single-chain variable fragment (scFv) derived from an antibody. The targeting sequence is typically connected to intracellular domains by a hinge/transmembrane region, commonly derived from CD8 or IgG4. The intracellular domains may contain co-stimulatory domains such as CD80, CD86, 4-1BBL, IL-2Rbeta, OX40L and CD70 and/or CD28 linked to the cytoplasmic signaling domain of CD3zeta.

Whole blood is composed of plasma, red blood cells (RBCs; or erythrocytes), platelets, and nucleated white blood cells, also referred to as leukocytes. The leukocytes can be further categorized into mononuclear cells and polymorphonuclear cells (or granulocytes). There are different techniques to obtain peripheral blood mononuclear cells (PBMCs), polymorphonuclear cells, leukocytes, or specific cell subsets, e.g., isolate specific cells directly by using flow cytometry, depleting red blood cells, centrifugation, and/or apheresis. Peripheral blood mononuclear cells (PBMCs) may be isolated by leukapheresis. T cells can be enriched by mononuclear cells counter-flow elutriation and expanded by addition of anti-CD3/CD28 antibody coated paramagnetic beads for activation of T cells. Cells may be expanded, harvested, and cryopreserved in infusible medium sometime after the subject has had an allogeneic stem-cell transplantation. Cells may be obtained by isolation from peripheral blood and optionally purified by fluorescent activated cells sorting e.g., mixing cells with fluorescent antibodies or other fluorescent agents (molecular beacons) and separating the cells by flow cytometry based fluorescent sorting.

CD3 is expressed on T cells as it is associated with the T cells receptor (TCR). The majority of TCR are made up of alpha beta chains (alpha beta T-cells). Alpha beta T-cells typically become double-positive intermediates (CD4+CD8+) which mature into single-positive (CD4+CD8−) T helper cells or (CD4-CD8+) cytotoxic T cells. T helper cells interact with antigen presenting dendritic cells and B cells. Upon activation with cognate antigen by dendritic cells, antigen specific CD4 T cells can differentiate to become various types of effector CD4 T cells with specific roles in promoting immune responses.

T cells may be isolated and separated from a human sample (blood or PBMCs or bone marrow) based on the expression of alpha beta T cells receptor (TCR), gamma delta T cells receptor, CD2, CD3, CD4, CD8, CD4 and CD8, NK1.1, CD4 and CD25 and other combinations based on positive or negative selection.

In a typical procedure, T cells are purified and isolated from blood or bone marrow. For example, T cells are collected via apheresis, a process that withdraws blood from the body and removes one or more blood components (such as plasma, platelets, or other white blood cells). The remaining blood is then returned into the body. The cells are exposed to a recombinant vector, such as a lentiviral or retroviral vector, that infects the cells in a way that a chimeric antigen receptor (CAR) protein is produced and presented in the cell membrane.

Bergamaschi et al. report circulating IL-15 exists as heterodimeric complex with soluble IL-15Ralpha in human and mouse serum. Blood. 2012, 120(1):e1-e8. See also Dubois et al. The Journal of Immunology, 2008, 180: 2099-2106. Bessard et al. report a fusion protein RLI, composed of the NH2-terminal (amino acids 1-77, sushi+) domain of IL-15 receptor a coupled via a linker to IL-15, and shown that it displayed better efficacy than IL-15 in vitro. Thus, in certain embodiments, providing an IL-15 and IL-15Ralpha binding complex coated on the particle may include or be substituted with the RLI fusion protein.

As used herein, unless the context suggests otherwise, “separating” refers to purify the cells from other cells or impurities that do not contain or contain less of a target molecule on the surface. One method of selecting proteins that are on the outside of a cell is to provide a specific binding agent, such as a primary antibody, and further trap the primarily antibody bound to the cell using a secondary antibody that is conjugated to magnetic beads. The magnetic beads can be captured by a magnetic field and separated from the rest of a solution. In another method, secondary antibodies contain a fluorescent marker, and the cells can be separated using flow cytometry or fluorescence activated sorting.

One can conjugate the cells with a fluorescent molecule to “stain” the cells within a sample. As these cells passed through the stream, the laser light would excite the fluorescent tag, or fluorochrome, which would emit photons of light at a higher wavelength (e.g. fluorescein isothiocyanate emits light at about 530 nm when excited by a 488 nm laser). This light can be collected and used to further categorize the cells.

The term “fluorescence-activated sorting,” “fluorescence-activated cell sorting” or “FACS” refers to a flow cytometry method of sorting a mixture of cells into two or more areas, typically one particle or cell at a time, based upon the fluorescent characteristics of each particle or cell. It is typically accomplished by applying an electrical charge and separating by movement through an electrostatic field. Typically, a vibrating mechanism causes a stream of cells to break into individual droplets. Just prior to droplet formation, a particle or cell in a fluid pass through an area for measuring fluorescence. An electrical charging mechanism is configured at the point where the stream breaks into droplets. Based on the fluorescence intensity measurement, a respective electrical charge is imposed on the droplet as it breaks from the stream. The charged droplets then move through an electrostatic deflection system that diverts droplets into areas based upon their relative charge. In some systems, the charge is applied directly to the stream, and the droplet breaking off retains charge of the same sign as the stream. In other systems, a charge is provided on a conduit inducing an opposite charge on the droplet.

### Multi-Cytokine Binding Particles, Libraries, and Uses in Cell Stimulation

This disclosure relates to libraries of multi-cytokine binding particles or zones and uses in method for screening cells for improved immune modulating functionality. In certain embodiments, the cells express a chimeric antigen receptor (CAR). In certain embodiments, the libraries are used to identify desirable particles or zones for stimulating cells useful for treating cancer and other therapies.

In certain embodiments, the cells are immune cells. In certain embodiments, the cells are T cells. In certain embodiments, the cells are natural killer cells. In certain embodiments, the T cell subsets have a greater proliferative capacity, are naive T cells, stem cell memory T (TSCM) cells, and/or central memory T (TCM) cells.

In certain embodiments, this disclosure relates to paramagnetic particles or zones coated with specific binding agents that specifically bind molecular targets.

In certain embodiments, this disclosure relates to paramagnetic particles/beads or zones coated with human fragment crystallizable (Fc) region-containing proteins that specifically bind molecular targets. In certain embodiments, the proteins are or are derived from variable light or heavy antibody sequences. In certain embodiments, the coated particles/beads or zones are used in methods for identifying optimal cell activation and expansion.

In certain embodiments, this disclosure relates to particles or zones comprising a coating with a specific binding agent that binds CD3, a specific binding agent that binds CD28, and a specific binding agent that binds a first cytokine, wherein the specific binding agent is complexed with the first cytokine. In certain embodiments, the specific binding agent that binds a first cytokine is a specific binding agent that binds IL-2. In certain embodiments, the specific binding agent that binds IL-2 is an anti-IL-2 antibody wherein the anti-IL-2 antibody is binding IL-2 providing an IL-2 antibody binding complex coated on the particle or zones. In certain embodiments, the coating further comprises a specific binding agent that binds a second cytokine, and the specific binding agent is complexed with the second cytokine, wherein the first cytokine and the second cytokine are not the same. In certain embodiments, the specific binding agent that binds a second cytokine is a specific binding agent that binds IL-15. In certain embodiments, the specific binding agent that binds IL-15 is an extracellular domain of IL-15Ralpha and wherein the extracellular domain of IL-15Ralpha is binding IL-15 providing an IL-15 and IL-15Ralpha binding complex coated on the particle or zone.

In certain embodiments, the surface ratios by weight or molecular distribution are about the same as provided on particle #16 which is about between 9 to 11 for a specific binding agent that binds CD3, about between 300 to 310 for a specific binding agent that binds CD28, about between 0 to 1 for a specific binding agent that binds IL-2, and about between 5 to 7 for a specific binding agent that binds IL-15.

In certain embodiments, the surface ratios by weight or molecular distribution are about the same as provided on particle #27 which is about between 0.5 to 2 for a specific binding agent that binds CD3, about between 300 to 310 for a specific binding agent that binds CD28, about between 0.5 to 2 for a specific binding agent that binds IL-2, and about between 0.0 to 0.5 for a specific binding agent that binds IL-15.

In certain embodiments, this disclosure relates to particles or zones comprising a coating with a specific binding agent that binds CD3, a specific binding agent that binds CD28, and a specific binding agent that binds a first cytokine, wherein the specific binding agent is complexed with the first cytokine. In certain embodiments, the specific binding agent that binds a first cytokine is a specific binding agent that binds IL-2, and/or IL-15, and/or IL-7. In certain embodiments, the specific binding agent that binds IL-2 is an anti-IL-2 antibody wherein the anti-IL-2 antibody is binding IL-2, and/or IL-15, and/or IL-7 providing an IL-2, and/or IL-15, and/or IL-7 antibody binding complex coated on the particle or zones.

In certain embodiments, this disclosure relates to methods of stimulating immune cells comprising contacting particles or zones as disclosed herein with an immune cell providing phenotypic change in the immune cell. In certain embodiments, the particles have an average diameter of between about 500 nm to 2000 nm. In certain embodiments, the ratio of cells to particles is about 1:5 or 1:10 or between 1:5 and 1:25. In certain embodiments, the phenotypic change in the immune cell is increased CD62L+ cell expression. In certain embodiments, the phenotypic change in the immune cell is increased clustering. In certain embodiments, the phenotypic change in the immune cell is increased CAR cell expression. In certain embodiments, contacting a particle or zone as reported herein with an immune cell is in in the absence of exogenous IL-2. In certain embodiments, contacting a particle or zone as reported herein with an immune cell is in a growth medium.

In certain embodiments, this disclosure relates to methods of stimulating T cells comprising contacting a particle or zone as disclosed herein with a T cell providing phenotypic change in the T cell. In certain embodiments, the particles have an average diameter of between about 500 nm to 2000 nm. In certain embodiments, the ratio of cells to particles is about 1:5 or 1:10 or between 1:5 and 1:25. In certain embodiments, the phenotypic change in the T cell is increased CD62L+ CAR T cell expression. In certain embodiments, the phenotypic change in the T cell is increased clustering. In certain embodiments, the phenotypic change in the T cell is increased CAR T cell expression. In certain embodiments, contacting a particle or surface as reported herein with a T cell is in in the absence of exogenous IL-2. In certain embodiments, contacting a particle or surface as reported herein with a T cell is in a growth medium.

In certain embodiments, this disclosure relates to libraries of particles or surfaces comprising a plurality of zones, wherein each of the particles or zones are coated with a plurality of specific binding agents that bind CD3, CD28, a first cytokine, and a second cytokine, wherein more than 3, 4, 5, 6, 7, 8, 9, or 10 of the particles or zones are coated with unique concentrations of the plurality of specific binding agents that bind CD3, CD28, a first cytokine, and a second cytokine.

In certain embodiments, this disclosure relates to libraries of particles or surfaces comprising a plurality of zones, wherein each of the particles or zones are coated with a plurality of specific binding agents that bind CD3, CD28, and a first cytokine, wherein 3, 4, 5, or more of the particles or zones coated with unique concentrations of the plurality of specific binding agents. In certain embodiments, the first cytokine is IL-2, wherein the specific binding agent that binds IL-2 is an anti-IL-2 antibody and wherein the anti-IL-2 antibody is binding IL-2 providing an IL-2 antibody binding complex coated on the particle or surface. In certain embodiments, the particles or zones further comprise a second cytokine. In certain embodiments, the second cytokine is IL-15, wherein the specific binding agent that binds IL-15 is an extracellular domain of IL-15Ralpha and wherein the extracellular domain of IL-15Ralpha is binding IL-15 providing an IL-15 and IL-15Ralpha binding complex coated on the particle or zone.

In certain embodiments, this disclosure relates to libraries of particles or surfaces comprising a plurality of zones, wherein each of the particles or zones are coated with a plurality of specific binding agents, wherein one of the plurality of specific binding agents in each of the particles or zones is a specific binding agent that binds CD3, wherein one of the plurality of specific binding agents in each of the particles or zones is a specific binding agent that binds CD28, wherein one of the plurality of specific binding agents in each of the particles or zones is a specific binding agent that binds a first cytokine, wherein one of the plurality of specific binding agents in each of the particles or zones is a specific binding agent that binds a second cytokine, wherein the first cytokine and the second cytokine are not the same molecular entity, wherein one of the plurality of particles or zones is coated with a specific binding agent that binds CD3 coated at a first percentage, wherein one of the plurality of particles or zones is coated with a specific binding agent that binds CD3 coated at a second percentage, wherein the first percentage and the second percentage are not the same percentage.

In certain embodiments, this disclosure relates to libraries of particles or surfaces comprising a plurality of zones, as reported herein, wherein one of the plurality of particles or zones is coated with a specific binding agent that binds CD28 coated at a first percentage, wherein one of the plurality of particles or zones is coated with a specific binding agent that binds CD28 coated at a second percentage, wherein the first percentage and the second percentage are not the same percentage.

In certain embodiments, this disclosure relates to libraries of particles or surfaces comprising a plurality of zones, as reported herein, wherein one of the plurality of zones is coated with a specific binding agent that binds the first cytokine coated at a first percentage, wherein one of the plurality of zones is coated with a specific binding agent that binds the first cytokine coated at a second percentage, wherein the first percentage and the second percentage are not the same percentage.

In certain embodiments, this disclosure relates to libraries of particles or surfaces comprising a plurality of zones, as reported herein, wherein one of the plurality of particles or zones is coated with a specific binding agent that binds the second cytokine coated at a first percentage, wherein one of the plurality of particles or zones is coated with a specific binding agent that binds the second cytokine coated at a second percentage, wherein the first percentage and the second percentage are not the same percentage.

In certain embodiments, this disclosure relates to methods testing whether cells are activated or proliferate upon exposure to coated particles or surfaces disclosed herein comprising contacting particles or surfaces disclosed herein coated with specific binding agents disclosed herein; and detecting, measuring, or quantifying changes in cellular properties, expression profiles, or proliferation characteristics.

In certain embodiments, this disclosure relates to method of screening, identifying or selecting optimal particles or surfaces for managing cancer or an immune therapy comprising, obtaining a sample comprising immune cells from a subject; contacting the sample with particles or zones in libraries as disclosed herein; detecting, measuring, quantifying, or observing a phenotypic change in the immune cells; and selecting particles or surfaces with an optimal phenotypic change for use in stimulating the immune cells for use in a cancer or immune cell therapy.

In certain embodiments, this disclosure relates to methods testing whether cells are activated or proliferate upon exposure to coated particles or zones disclosed herein comprising contacting particles or zones disclosed herein coated with specific binding agents disclosed herein; and detecting, measuring, or quantifying changes in cellular properties, expression profiles, or proliferation characteristics.

### Methods in Treating Cancer

In certain embodiments, this disclosure relates to methods of treating cancer or other immune cell disease or condition comprising, isolating immune cells from a subject diagnosed with cancer or other immune cell disease or condition, contacting the isolated immune cells with particles or surfaces as disclosed herein providing activated immune cells; contacting the activated immune cells with a recombinant vector expressing a chimeric antigen receptor providing chimeric antigen receptor expressing activated immune cells; and administering an effective amount of chimeric antigen receptor expressing activated immune cells to a subject in need thereof. In certain embodiments, the cancer is a hematological cancer. In certain embodiments, the cancer is a metastatic cancer. In certain embodiments, the cancer is a tumor. In certain embodiments, administering is in combination with administering an additional anticancer agent or active agent.

In certain embodiments, this disclosure relates to methods of treating cancer comprising, isolating T cells from a subject diagnosed with cancer, contacting the T cell with particles or surfaces as reported herein providing activated cells; contacting the activated cells with a recombinant vector expressing a chimeric antigen receptor providing chimeric antigen receptor expressing activated immune cells; and administering an effective amount of chimeric antigen receptor expressing activated immune cells to a subject in need thereof.

In certain embodiments, the cell is an immune cell such as subsets of T cells, B cells, NK cells, monocytes, macrophages, or combinations thereof.

In certain embodiments, the T cells are produced, selected, and/or purified to maximize the amount of CD8+ T cells.

In certain embodiments, the T cells are produced, selected, and/or purified to maximize the amount of CD4+ T cells.

In certain embodiments, the T cells are produced, selected, and/or purified to maximize at a defined 1:1 CD4+ to CD8+ T cell ratio.

In certain embodiments, the cells express a chimeric antigen receptor and optionally macrophage colony-stimulating factor 1 receptor (CSF-1R).

In certain embodiments, the cancer is a hematological cancer, metastatic cancer, or tumor.

In certain embodiments, the activated cells are administered is in combination with an additional chemotherapy agent. In certain embodiments, the additional chemotherapy agent is a checkpoint inhibitor.

In certain embodiments, administering is implanting, injecting the activated cells in proximity of a tumor or tissue comprising cancerous cells, e.g., inside or within 1, 2, or 3 cm of a solid tumor mass or tissue containing cancerous cells.

In certain embodiments, the cells are infused directly to tumors at various anatomical sites, such as the brain, breast, thorax, lung, and liver.

In certain embodiments, the cells are unmodified or genetically engineered. In certain embodiments, the immune cells express a chimeric antigen receptor. In certain embodiments, the recombinant gene construct is a recombinant vector, virus, lentivirus, or retrovirus, and virally or non-virally introduced into immune cells, e.g., introduced into immune cells with a retrovirus, introduced into immune cells with transposon-based genomic integration, introduced into immune cells with electroporation, introduced into immune cells with mechanoporation.

In certain embodiments, the chimeric antigen receptor specifically binds (EGFR) epidermal growth factor receptor, (HER2) human epidermal growth factor receptor 2, (MUC1) mucin1, (MUC16) mucin16, (EpCAM) epithelial cell adhesion molecule, (AFP) alpha-fetoprotein, (FAP) familial adenomatous polyposis, (CEA) carcinoembryonic antigen, (PSCA) prostate stem cell antigen, (PSMA) prostate-specific membrane antigen, (PSA) prostate-specific antigen, (AXL) AXL receptor tyrosine kinase, (DLL3) delta-like 3, (EPHA2) EPH receptor A2, (FRα) folate receptor alpha, (LMP1) Epstein-Barr virus latent membrane protein 1, (MAGE) melanoma antigen gene protein, MAGE-A1, MAGE-A3, MAGE-A4, (DR5) death receptor 5, (NKG2D) natural killer group 2 member D receptor, (CAIX) carbonic anhydrase IX, (TAG-72) tumor-associated glycoprotein 72, (GUCY2C) guanylate cyclase 2C, (ANTXR1) anthrax toxin receptor 1, (GSPG4) general secretion pathway protein G, (ROR) RAR-related orphan receptors, IL13RA2 (Interleukin 13 Receptor Subunit Alpha 2), Wilms' tumor 1 (WT1), Survivin, Tn (aGalNAc-O-Ser/Thr), sialyl-Tn (aNeuAc2,6-aGalNAc-O-Ser/Thr), TF (bGall,3-aGalNAc-O-Ser/Thr), CA 19-9 (Neu5Acα2-3Galβ1-3[Fucα1-4]GlcNAcβ), Telomerase reverse transcriptase (TERT), Beta-hCG (Human chorionic gonadotropin), p53, Ras, bladder tumor antigen (BTA), antibody specific antigen Om5, GD2 (Ganglioside GD2), integrin alpha-v/beta-6, or mesothelin antigen. In certain embodiments, the chimeric antigen receptor is an antibody single-chain variable fragment (scFv).

In certain embodiments, immune cells or T cells produced by methods disclosed herein are used in methods for treating a subject diagnosed with cancer. In certain embodiments, the cancer is a hematological malignancy such as acute lymphoblastic leukemia (ALL), acute myelogenous leukemia (AML), chronic lymphocytic leukemia (CLL), small lymphocytic lymphoma (SLL), chronic myelogenous leukemia, acute monocytic leukemia (AMOL), chronic myeloid leukemia (CML), myeloproliferative neoplasms (MPNs), and lymphomas, Hodgkin's lymphomas, and non-Hodgkin's lymphomas such as Burkitt lymphoma, B-cell lymphoma.

In certain embodiments, the cancer is a solid tumor, cellular malignancy, or hematological malignancy. In certain embodiments, the cancer is lung cancer, non-small cell lung cancer, small cell lung cancer, bronchus cancer, mesothelioma, malignant pleural mesothelioma, lung adenocarcinoma, breast cancer, prostate cancer, colon cancer, rectum cancer, colorectal cancer, gastrointestinal cancer, stomach cancer, esophageal cancer, ovarian cancer, cervical cancer, melanoma, kidney cancer, pancreatic cancer, pancreatic ductal adenocarcinoma (PDA), thyroid cancer, brain cancer, glioblastoma (GBM), medulloblastoma, glioma, neuroblastoma, liver cancer, bladder cancer, uterine cancer, bone cancer, osteosarcoma, sarcoma, rhabdomyosarcoma, Ewing's sarcoma, retinoblastoma, nasopharyngeal carcinoma.

In certain embodiments, the immune cells or T cells made by methods disclosed herein are used for treating cancer and are administered in combination with another anticancer agent. In certain embodiments, the anticancer agent is abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, tisagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof such as cyclophosphamide, methotrexate, 5-fluorouracil (CMF); doxorubicin, cyclophosphamide (AC); mustine, vincristine, procarbazine, prednisolone (MOPP); adriamycin, bleomycin, vinblastine, dacarbazine (ABVD); cyclophosphamide, doxorubicin, vincristine, prednisolone (CHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC). In certain embodiments, the anticancer agent is an anti-PD-1, anti-PD-L1 anti-CTLA4 antibody or combinations thereof, such as an anti-CTLA4 (e.g., ipilimumab, tremelimumab) and anti-PD1 (e.g., nivolumab, pembrolizumab, cemiplimab) and anti-PD-L1 (e.g., atezolizumab, avelumab, durvalumab).

In certain embodiments, the cells are administered to a subject with a lymphodepleted environment due to prior or concurrent administration of lymphodepleting agents such as cyclophosphamide and fludarabine).

### EXAMPLES

### Multi-Cytokine Backpack (MCBs)

The core scaffold for MCBs are protein G conjugated iron oxide microparticles that are about 1 micron in diameter. Via Fc-protein G affinity, any protein with an Fc domain can be incubated with and conjugated onto these microparticles. For synthesis of cytokine-loaded microparticles, iron oxide particles were added to wells of a standard 96-well plate, then incubated with a protein cocktail of aCD3/aCD28/aIL-2/IL-15Ra-Fc chimera protein which are labeled with fluorescent tags, allowing for downstream characterization of protein abundance per particle. Following incubation, protein-bound particles were then purified from unbound protein via magnetic field-induced sedimentation through use of a 96 well plate magnet. Post-synthesis abundance of aCD3/aCD28/aIL-2/IL-15Ra-Fc on particles was assessed via fluorescence and absorbance measurements on a standard plate reader. Fluorescence measurements allowed for quantification of protein concentration in post-synthesis product, while absorbance measurements allowed for quantification of particle concentration. IL-2 and IL-15 cytokines were subsequently conjugated to protein-bound particles via incubation and purification in a similar manner.

A compositionally diverse library of T cell-engaging microparticles was assembled. Micro-particle formulations were screened and prioritized based on CAR T cell manufacturing and phenotypic screening. Heatmaps of micro-particle cytokines within short-listed Multi-Cytokine Backpack formulations were generated.

### Multi-Cytokine Backpack CAR T Cells Promote the Expansion of CAR+CD8+ T Cells and Demonstrate Unique Phenotypes

Particles were recreated using solutions containing the ratios of each of the binding agents, by weight, of the antibodies to each other.

Table 1 shows specific particles with surface ratios of binding agents relative to each component

| | aCD3 | aCD28 | aIL2 | IL15RaFc |
|---|---|---|---|---|
| 9 | 10.00 | 305.23 | 21.35 | 0.06 |
| 16 | 10.00 | 305.23 | 0.00 | 6.30 |
| 27 | 1.00 | 305.23 | 1.07 | 0.00 |
| 43 | 5.00 | 305.23 | 1.07 | 0.63 |

The values in the table are the weight ratios (μg) of each antibody added to the beads in solution.

MCB formulations successfully transduce human primary T cells. MCB formulations promote the expansion of CAR+CD8+ T cells compared to traditional DynaBeads' manufacturing. MCBs with additional cytokine support demonstrate enhanced production of IL-2 post CAR T cell manufacturing. CAR T cells manufactured with MCB formulations demonstrate comparable SCM/N phenotype. MCB formulations lead to unique exhaustion phenotype profile. CAR T cells (anti-hCD19) manufactured with MCB lyse antigen-relevant Raji GFPffLuc cells at varying E:T ratios were examined. CAR T cells (anti-hCD19) manufactured with MCB produce enhanced levels of IL-2 in the presence of CD19 antigen.

Multi-Cytokine Backpack Formulations successfully transduce DLBCL patient T cells to enhance product phenotype. MCB formulations promotes the expansion of CAR+CD8+ T cells of DLBCL patients. DLBCL patient CAR T cells manufactured with MCB demonstrated patient-dependent enhanced SCM/N phenotype compared to CAR T cells manufactured with DynaBead™. MCB manufactured DLBCL patient CAR T cells display a unique exhaustion phenotype.

Unique multi-cytokine backpack formulations successfully transduce both healthy primary T cells and DLBCL patient T cells. CAR T cells manufactured with multi-cytokine backpack formulations exhibit enhanced CD8+ populations and unique exhaustion phenotypes compared to T cells manufactured traditionally with DynaBeads™ and IL-2.

What is claimed is:

1. A particle comprising a coating with
a specific binding agent that binds CD3,
a specific binding agent that binds CD28, and
a specific binding agent that binds a first cytokine, wherein the specific binding agent is complexed with the first cytokine.

2. The particle of claim 1, wherein the specific binding agent that binds a first cytokine is a specific binding agent that binds IL-2.

3. The particle of claim 2, wherein the specific binding agent that binds IL-2 is an anti-IL-2 antibody and wherein the anti-IL-2 antibody is binding IL-2 providing an IL-2 antibody binding complex coated on the particle.

4. The particle of claim 1, wherein the coating further comprises a specific binding agent that binds a second cytokine, and the specific binding agent is complexed with the second cytokine, wherein the first cytokine and the second cytokine are not the same.

5. The particle of claim 4, wherein the specific binding agent that binds a second cytokine is a specific binding agent that binds IL-15.

6. The particle of claim 5, wherein the specific binding agent that binds IL-15 is an extracellular domain of IL-15Ralpha and wherein the extracellular domain of IL-15Ralpha is binding IL-15 providing an IL-15 and IL-15Ralpha binding complex coated on the particle.

7. A method of stimulating immune cells comprising contacting a particle of claim 1 with an immune cell providing phenotypic change in the immune cell.

8. The method of claim 7 wherein the phenotypic change in the immune cell is increased CD62L+ cell expression.

9. The method of claim 7 wherein the cell to bead ration is between 1:5 and 1:25.

10. The method of claim 7, wherein contacting the particle with an immune cell is in in the absence of exogenous IL-2.

11. A method of treating cancer or other immune cell disease or condition comprising,
contacting immune cell with particles of claim 1 providing activated immune cells;
contacting, transfecting, or inserting into the activated immune cells a recombinant vector encoding a chimeric antigen receptor providing chimeric antigen receptor expressing activated immune cells; and
administering an effective amount of chimeric antigen receptor expressing activated immune cells to a subject in need thereof.

12. The method of claim 11, wherein the cancer is a hematological cancer.

13. The method of claim 11, wherein the cancer is a metastatic cancer.

14. The method of claim 11, wherein the cancer is a tumor.

15. The method of claim 11, wherein administering is in combination with administering an additional anticancer agent or active agent.

16. A library of particles comprising
a plurality of zones, wherein each of the zones comprise particles coated with a plurality of specific binding agents that bind CD3, CD28, and a first cytokine,
wherein more than 3 of the zones comprise particles coated with unique concentrations of the plurality of specific binding agents.

17. The library of particles of claim 16, wherein the first cytokine is IL-2, wherein the specific binding agent that binds IL-2 is an anti-IL-2 antibody and wherein the anti-IL-2 antibody is binding IL-2 providing an IL-2 antibody binding complex coated on the particle.

18. The library of particles of claim 17, further comprising a second cytokine.

19. The library of particles of claim 18, wherein the second cytokine is IL-15, wherein the specific binding agent that binds IL-15 is an extracellular domain of IL-15Ralpha and wherein the extracellular domain of IL-15Ralpha is binding IL-15 providing an IL-15 and IL-15Ralpha binding complex coated on the particle.

20. A method of selecting optimal particles for an immune therapy comprising,
obtaining a sample comprising immune cells from a subject;
contacting the sample with particles in zones of the library of claim 16;
detecting, measuring, quantifying, or observing a phenotypic change in the immune cells;
and
selecting particles with an optimal phenotypic change for use in stimulating the immune cells for use in an immune cell therapy.
